(19) United States
(12) Patent Application Publication
(10) Pub. No.: US 2024/0295472 A1
(43) Pub. Date: Sep. 5, 2024

(54) SAMPLING STATION AND VALVE USEFUL THEREIN

(71) Applicant: KUPFERLE, LLC, ST. LOUIS, MO (US)

(72) Inventors: Kyle J. WILDE, Hillsboro, MO (US); Daniel C. LORENTZ, Des Peres, MO (US)

(21) Appl. No.: 18/593,287

(22) Filed: Mar. 1, 2024

Related U.S. Application Data
(60) Provisional application No. 63/449,865, filed on Mar. 3, 2023.

(51) Int. Cl. G01N 1/20; E03B 7/07; G01N 1/10; G01N 33/18

(52) U.S. Cl. CPC G01N 1/2035; E03B 7/07; G01N 33/18; G01N 2001/1031; G01N 2001/205

(57) ABSTRACT

A hydrant includes a standpipe, a discharge nozzle, and a subterranean valve operated by a valve stem. The operating stem is hollow and has an inlet at its lower end and a drain outlet at its upper end, thereby providing a path to flush the sampling station when the subterranean valve is shut and the discharge nozzle and drain outlet are open by applying a differential pressure. The subterranean valve includes a cartridge having a fixed portion and a moveable portion. The fixed portion is fixed to the end of the stem and turns with it but does not move axially. The moveable portion moves axially but does not rotate. The fixed and moveable portions are connected to each other by a threaded connector. The cartridge, including a valve plug, a valve seat, and a threaded operating mechanism can be removed simply by removing a top cap on the standpipe.

### CROSS-REFERENCE TO RELATED APPLICATIONS

Priority is claimed to U.S. provisional application No. 63/449,865, filed Mar. 3, 2023, the entire contents, including the Appendices, of which are hereby incorporated by reference.

### STATEMENT REGARDING FEDERALLY SPONSORED RESEARCH

Not applicable.

### FIELD

This disclosure relates to water hydrants, and in particular to sampling stations. It also relates to valves useful therein and in other hard-to-reach locations.

### BACKGROUND

Sampling stations are widely used for sampling water in subterranean water distribution systems to assess for chemical and biologic impurities. Some designs of sampling stations use a subterranean valve operated through an operating stem extending above ground to a manually operated handle. In temperate climates, these hydrants must be protected against freezing. To preserve the sampled water from contamination by ground water, these hydrants eliminate the usual subterranean drain holes, even those with check valves.

Examples of commercially available sampling stations are the Kupferle Eclipse® #88-SS sampling station available from John C. Kupferle Foundry Company, St. Louis, Missouri, and the Hydro-Guard® BSS-01/02 sampling station available from Mueller Water Products, Inc., Atlanta, Georgia. In the Kupferle sampling station, the stem extends through the standpipe that delivers the sample to be tested. In the Hydro-Guard device, the stem extends through a separate curb stop upstream from the standpipe.

To prevent freezing without the use of a subsurface drain which might lead to contamination by water external to the sampling station, these sampling stations utilize an evacuation pipe running up the outside of the standpipe. Attaching a vacuum pump to the upper end of the evacuation pipe, and opening a valve at its upper end, draws water from the bottom of the standpipe, leaving the standpipe empty, or at least empty to a depth below the frostline. Attaching a pressurized gas source, such as pressurized air, to the evacuation pipe or standpipe outlet can also substantially empty the standpipe. The evacuation pipe is of smaller diameter than the standpipe and is therefore subject to damage and to flash freezing. It also adds complication to installation and servicing of the sampling station, and if not emptied may itself be subject to freezing.

A sanitary hydrant which addresses the same freezing problem by providing a subterranean reservoir into which its subterranean drain discharges is sold by Woodford Manufacturing Company, Colorado Springs, Colorado (a division of WCM Industries, Inc.) as its Model S3 and is described in Ball, et al., U.S. Pat. RE47,789.

Because the Kupferle 88SS and the Woodford S3 hydrants include a plug-and-seat closure, the operating screw threads at the upper end of the operating stem are also subject to freezing.

Snow-making hydrants are also subject to freezing problem. Such hydrants sold by the Roger's Hydrant Company (a division of WCM Industries, Inc.). Colorado Springs, Colorado, place the operating screw below the frost line, threaded into the inlet casting. A subterranean drain is used.

Another snow-making hydrant is sold by HTM Hydro-Tech Manufacturing Inc., West Kelowna, British Columbia, Canada (HTM). HTM sells an internal drain H-Series and an external drain E-Series. These hydrants include a valve assembly which is threaded into a valve housing and can be unscrewed and removed from above using a special service wrench. The valve assembly includes a valve body, the lower surface of which may form a seal with a disc carried by a valve screw which is threaded into the valve body. The valve screw includes a passage for water into a riser pipe. The valve screw is connected to an operating shaft which rotates and moves vertically through a graphite foil packing at the upper end of the riser pipe.

Sampling stations are also used for other purposes, for example in industrial process lines. Those and other hydrants may be subject to similar problems and limitations.

Other devices also utilize plug-and-seat valves which are difficult to reach for servicing. Valves in accordance with the present invention may be beneficial in these devices.

### SUMMARY OF THE INVENTION

In accordance with one aspect of the present invention, the operating stem of a hydrant, such as a sampling station, is utilized as an evacuation tube by making it hollow and providing it with an inlet at its lower end and an outlet at its upper end. The outlet preferably is normally closed except when the hydrant is being evacuated.

In accordance with another aspect of the invention, the operating stem of a hydrant carries a valve cartridge at its lower, distal, end, the valve cartridge including a fixed portion and a moveable portion. The fixed portion is fixed to the end of the stem and turns with it but does not move axially. The moveable portion moves axially but does not rotate. The fixed and moveable portions are connected to each other by a threaded connector. In an embodiment, the fixed portion is a valve plug and carries a threaded extension rod; the moveable portion is a valve seat having an internally threaded through-hole receiving the threaded extension rod. In an embodiment, the moveable portion reciprocates in an inlet fitting having a cylindrical axial passageway with which a cylindrical part of the movable portion forms a watertight fit. A non-circular section of the inlet passageway interacts with a non-circular part of the moveable portion and prevents the moveable portion from rotating freely. The watertight fit is, in embodiments, provided by O-rings on the exterior of the circular part of the moveable portion.

The provision of a cartridge including the plug, the valve seat, and the threaded operating mechanism, makes possible the simple removal of all operating components of the valve for servicing. It also places all operating components of the valve below the frost line. It also reduces vibration of the operating stem because the operating stem is supported radially at both ends. In embodiments in which the valve plug is axially stationary and the valve seat moves axially, the valve closes with the water and the force of the pressurized water aids in turning off the flow. Because the valve stem turns but does not move axially, sealing the upper end of the valve stem is greatly simplified.

Other aspects of the present invention will become apparent in light of the following description of exemplary embodiments of the invention.

Although the novel constructions described herein are particularly advantageous as applied to sampling stations, their utility is not limited thereto.

### DESCRIPTION OF THE DRAWINGS

In the accompanying drawings which form part of the specification:

FIG. 1 is a view in side elevation of a sampling station according to one embodiment, showing a fixed assembly attached to a subterranean water system and a removable assembly disassembled from the fixed part.

FIG. 2 is a view in front elevation of the assembled sampling station of FIG. 1.

FIG. 3 is a sectional view of the sampling station of FIGS. 1 and 2, taken along line 3-3 of FIG. 2.

FIGS. 4 and 5 are sectional views of a lower portion of the sampling station of FIGS. 1-3, indicated by line 4-4 of FIG. 3, depicting a valve of the sampling station in an open and closed position, respectively.

FIG. 6 is an exploded view in cross-section showing components of a valve assembly of the sampling station of FIGS. 1-3.

FIG. 7 is a view in perspective of a valve body of the sampling station of FIGS. 1-3

FIG. 8 is a top plan view of the valve body of FIG. 7.

FIG. 9 is a view in perspective of a valve seat carrier (piston) of the sampling station of FIGS. 1-3.

FIG. 10 is a top plan view of the valve seat carrier of FIG. 9.

FIG. 11 is a bottom plan view of the valve seat carrier of FIG. 9.

FIG. 12 is a view in perspective of a valve core of the sampling station of FIGS. 1-3.

FIG. 13 is a top plan view of the valve core of FIG. 12.

FIG. 14 is a bottom plan view of the valve core of FIG. 12.

FIG. 15 is a sectional view of an upper portion of the sampling station of FIGS. 1-3, corresponding to FIG. 3 with a drain portion turned ninety degrees for clarity.

FIG. 16 is a view in perspective of a headstock of the sampling station of FIGS. 1-3

FIG. 17 is a view in perspective of a coupling nut of the sampling station of FIGS. 1-3

FIG. 18 is a view in perspective of a top cap of the sampling station of FIGS. 1-3.

FIG. 19 is a view in side elevation of the top cap of FIG. 18.

In the figures, corresponding reference characters and symbols indicate corresponding parts in the several views of the drawings.

### DETAILED DESCRIPTION

The following detailed description illustrates the disclosed systems and methods by way of example and not by way of limitation. The description enables one skilled in the art to make and use the disclosed apparatuses and the methods, describes several embodiments, adaptations, variations, alternatives, and uses of the same, including what is presently believed to be the best mode of making and using the apparatuses. Additionally, it is to be understood that the disclosed embodiments are not limited to the details of construction and the arrangements of components set forth in the following description or illustrated in the drawings. The claimed systems and/or methods are capable of other embodiments and of being practiced or being carried out in various ways. Also, it is to be understood that the phraseology and terminology used herein is for the purpose of description.

As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the invention and are not intended to limit the scope of the appended claims.

Unless otherwise defined, all technical and scientific terms used herein have the same meaning as commonly understood by one of ordinary skill in the art to which this disclosure belongs. The terminology used herein is for the purpose of describing particular example embodiments only and is not intended to be limiting. As used herein, the singular forms “a”, “an”, and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps can be employed.

When an element, object, device, apparatus, component, region or section, etc., is referred to as being “on”, “engaged to or with”, “connected to or with”, or “coupled to or with” another element, object, device, apparatus, component, region or section, etc., it can be directly on, engaged, connected or coupled to or with the other element, object, device, apparatus, component, region or section, etc., or intervening elements, objects, devices, apparatuses, components, regions or sections, etc., can be present. In contrast, when an element, object, device, apparatus, component, region or section, etc., is referred to as being “directly on”, “directly engaged to”, “directly connected to”, or “directly coupled to” another element, object, device, apparatus, component, region or section, etc., there may be no intervening elements, objects, devices, apparatuses, components, regions or sections, etc., present. Other words used to describe the relationship between elements, objects, devices, apparatuses, components, regions, or sections, etc., should be interpreted in a like fashion (e.g., “between” versus “directly between”, “adjacent” versus “directly adjacent”, etc.).

As used herein the phrase “coupled” will be understood to mean two or more elements, objects, devices, apparatuses, components, etc., that are directly or indirectly connected to each other in an operational and/or cooperative manner such that operation or function of at least one of the elements, objects, devices, apparatuses, components, etc., imparts or causes operation or function of at least one other of the elements, objects, devices, apparatuses, components, etc. Such imparting or causing of operation or function can be unilateral or bilateral. The phrase “in fluid communication” refers to two or more elements, objects, devices, apparatuses, components, etc., that are directly or indirectly connected to each other using a connection suitable for fluid (e.g., gas, liquid, and/or fluidized granular solids) to flow between each other, e.g., a pipe, duct, channel, or the like. There may be one or more intervening elements, objects, devices, apparatuses, components, etc. between elements, objects, devices, apparatuses, components, etc. in fluid communication in the case that such elements are indirectly connected to each other. It should be understood that elements, objects, devices, apparatuses, components, etc. that are in fluid communication and are depicted as being serially coupled (e.g., by a pipe) can in some embodiments be direct connections without intervening elements, objects, devices, apparatuses, components, etc. The term “hydrant” is used broadly to indicate an outlet structure from a fluid main from which fluid, such as water or fuel, can be tapped. The term “standpipe” is used broadly to indicate a generally vertical conduit extending from a subterranean fluid source to an outlet accessible from above ground.

As used herein, “line” or similar terminology should be understood to mean a connection for conveying fluids (e.g., gas, liquid, and/or fluidized granular solids) and is typically a pipe. Other suitable components can be used in alternative embodiments.

As used herein, the term “and/or” includes any and all combinations of one or more of the associated listed items. For example, A and/or B includes A alone, or B alone, or both A and B.

Although the terms first, second, third, etc. can be used herein to describe various elements, objects, devices, apparatuses, components, regions or sections, etc., these elements, objects, devices, apparatuses, components, regions or sections, etc., should not be limited by these terms. These terms may be used only to distinguish one element, object, device, apparatus, component, region or section, etc., from another element, object, device, apparatus, component, region or section, etc., and do not necessarily imply a sequence or order unless clearly indicated by the context.

Moreover, it will be understood that various directions such as “upper”, “lower”, “bottom”, “top”, “left”, “right”, “first”, “second” and so forth are made only with respect to explanation in conjunction with the drawings, and that components may be oriented differently, for instance, during transportation and manufacturing as well as operation. Because many varying and different embodiments may be made within the scope of the concept(s) taught herein, and because many modifications may be made in the embodiments described herein, it is to be understood that the details herein are to be interpreted as illustrative and non-limiting.

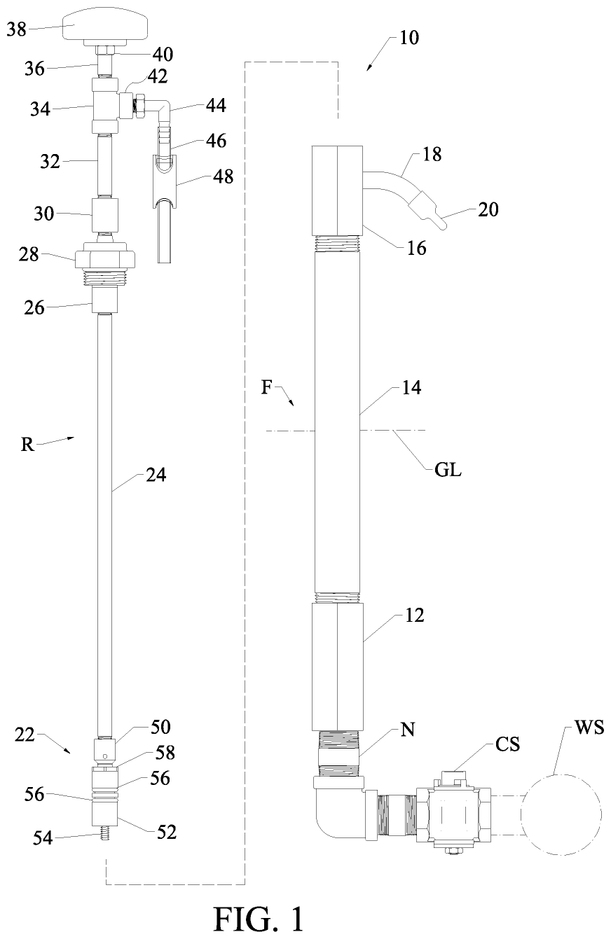

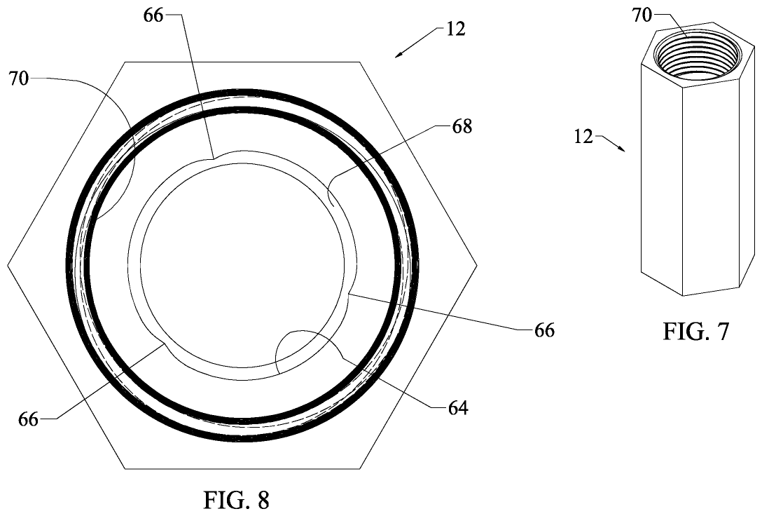

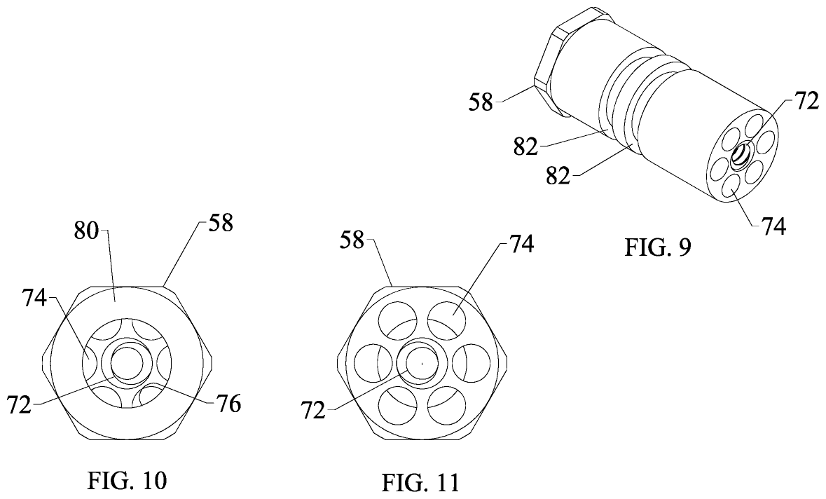

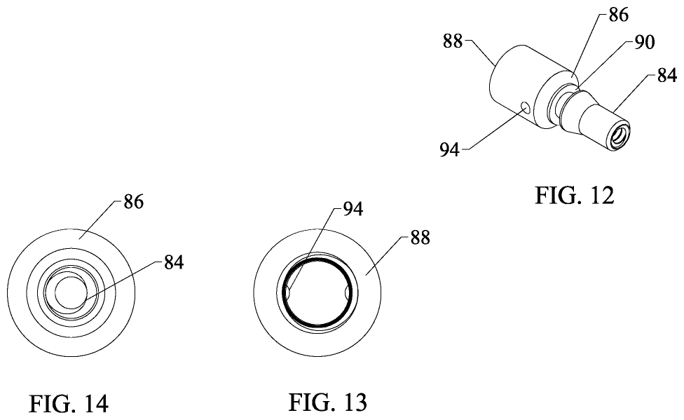

Referring now to FIGS. 1-19 for an illustrative embodiment, a sampling station 10 is a modification of a commercially available Kupferle Eclipse® 88SS sampling station. Like the Kupferle Eclipse® 88SS sampling station, all the wetted metal parts of the sampling station 10 are made of stainless steel.

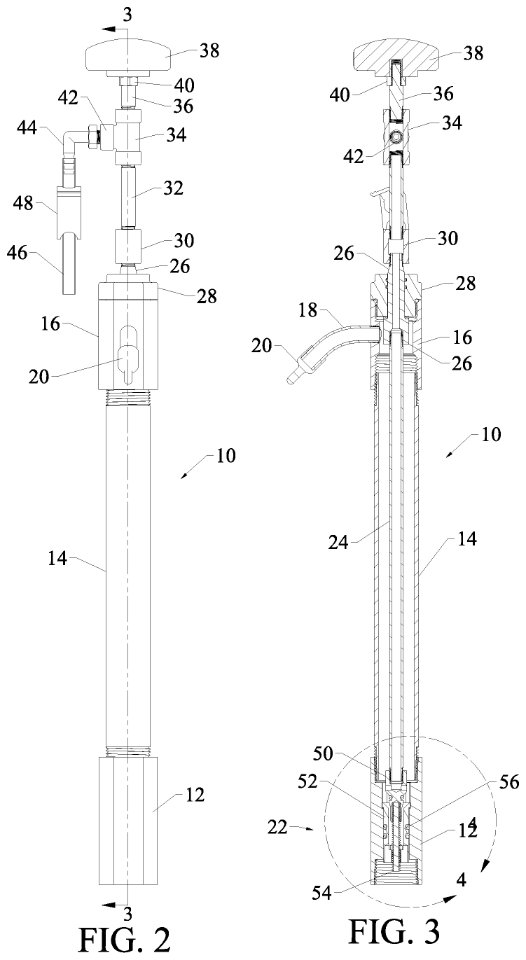

As shown in FIG. 1, the sampling station 10 includes a fixed assembly F and a removable assembly R. FIGS. 2 and 3 show the sampling station 10 assembled.

The fixed assembly F includes a valve body 12 threaded onto a first, lower or distal, end of a standpipe 14, and a headstock 16 threaded onto a second, upper or proximal, end of the standpipe 14. The lower end of the valve body 12 is internally threaded for coupling to a nipple N, attached through a curb stop CS to a water source WS, typically a subterranean water main. The curb stop CS is conventionally a valve, like a ball valve, operated from the surface using a suitable tool. Closing the curb stop CS is required only when the removable assembly R is to be removed for servicing. An outlet nozzle 18 is threaded into a wall of the headstock 16 and communicates through the head stock 16 with the standpipe 14. A nozzle cap 20 covers the outlet nozzle 18 when the sampling station valve is closed and the sampling station 10 is not being drained.

The removable assembly R includes a valve assembly 22 in the form of a valve cartridge threaded onto the lower end of a pipe forming a hollow valve stem 24, which acts as both a valve operation rod and a drain tube. A connecting nut 26 rotatably supporting a top cap 28 is threaded onto the upper end of the hollow valve stem 24. A pipe coupling 30 is threaded onto the upper end of the connecting nut 26. A pipe nipple 32 is threaded into the coupling 30. A tee 34 is threaded onto the pipe nipple 32, and a handle-mounting rod 36 is threaded into the upper end of the tee 34. A handle 38 is threaded onto the upper end of the handle-mounting rod 36 and is secured by a jam nut 40. Leg 42 of the tee 34 carries a ninety-degree barb 44 which carries a drain hose 46. Hose clamp 48 controls flow through the drain hose 46.

The lengths of the standpipe 14 and of the hollow valve stem 24 are chosen to place the valve assembly 22 below the frost line in the locale where the sampling station 10 is installed, with the headstock 16 above ground level GL a desired distance.

The valve assembly/valve cartridge 22 controls the flow of water through the standpipe 14 between the valve body 12 and the nozzle 18, thereby allowing for water to be sampled at the surface.

The drain hose 46 is used for draining the sampling station 10 as protection against freezing.

The valve cartridge 22 includes a valve core 50, a valve seat carrier piston 52 in the form of a piston, and an operating screw 54 controlling relative axial movement between the valve seat carrier piston 52 and valve core 50, hence controlling opening and closing of the valve core and valve seat. In this embodiment, the valve seat carrier piston 52 is sealed with the interior of the valve body 12 by O-rings 56 and is prevented from rotating freely by a hex head 58, as described hereinafter. The operating screw 54 is secured to the valve core 50 and is threaded through the valve seat carrier piston 52, so that turning the handle 38 moves the valve seat carrier piston 52 toward and away from the valve core 50, thereby closing and opening the valve cartridge 22, as described in more detail hereinafter.

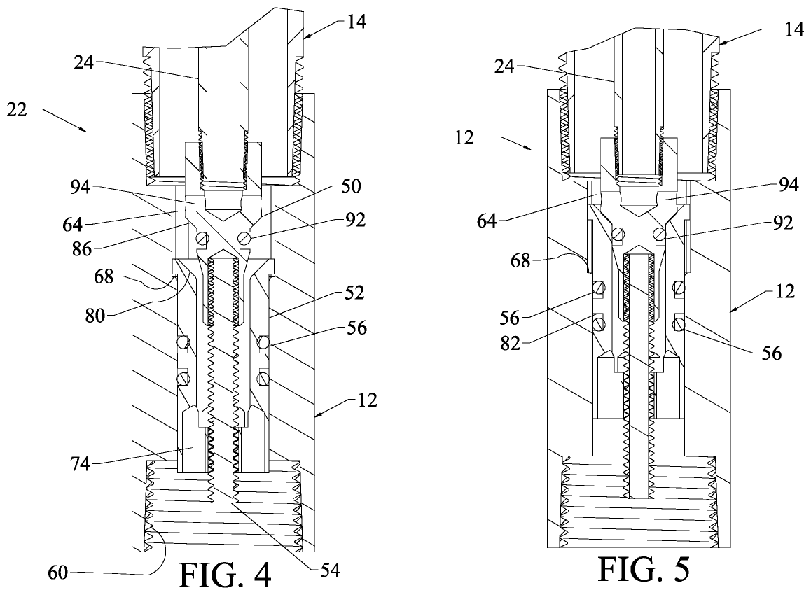

FIGS. 4-14 illustrate details of the lower, subterranean, portion of the sampling station 10.

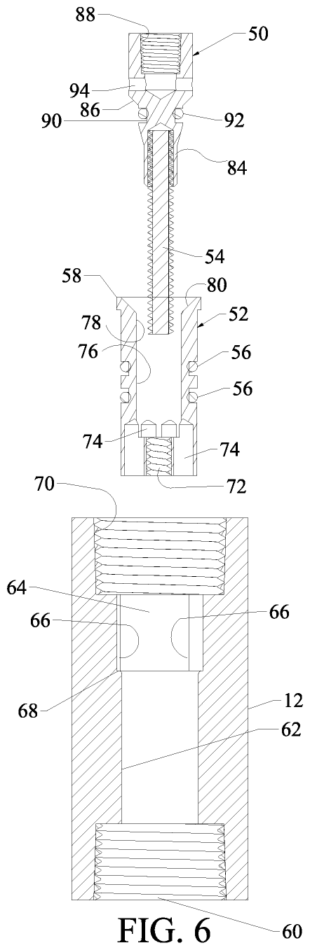

As shown in FIGS. 6-8, the valve body 12 has a hexagonal exterior. At its lower end, the valve body 12 is internally threaded as shown at 60 for connection to a subterranean water distribution system through nipple N. A cylindrical bore 62 is sized to form a fluid-tight sliding fit with the O-rings 56 on the cylindrical exterior of the valve seat carrier piston 52. At its upper end, the bore 62 includes an enlarged scalloped portion 64 sized to form a loose sliding fit with the hex head 58 of the valve seat carrier piston 52 while preventing free rotation of the valve seat carrier piston 52. As shown in FIG. 8, the enlarged scalloped portion 64 includes three protrusions 66 sized to prevent free rotation of the hex head 58. The protrusions allow a maximum of about 20° to 25° of rotation of the hex head 58 before it engages the protrusions 66. As also shown in FIG. 8 and in FIGS. 4-6, at the lower edge of enlarged scalloped portion 64 a shoulder 68 is sized to engage the lower edge of hex head 58 and limit downward movement of the valve seat carrier piston 52. At its upper end, the valve body 12 is internally threaded as shown at 70 to receive the threaded lower end of the standpipe 14.

As shown in FIGS. 4-6 and 9-11, the valve seat carrier piston 52 includes at its lower end a central threaded through-hole 72 into which the operating screw 54 is threaded when the valve cartridge 22 is assembled. Surrounding the central threaded through-hole 72 are six tubular channels 74 permitting water to flow from the bore 62 of the vale body 12 into a central bore 76 of the valve seat carrier piston 52. The upper portion of the central bore 76 forms a valve seat 78, above which the central bore 76 tapers outwardly to form a conical surface 80. As previously described, the exterior of the valve seat carrier piston 52 is generally cylindrical with an integral hex head 58 at its upper end. Two O-rings 56 sit in annular grooves 82 in the cylindrical outer surface of the valve seat carrier piston 52.

As shown in FIGS. 4-6 and 12-14, the valve core 50 includes an internally threaded lower cylindrical nose section 84, a conical tapered section 86, and an internally threaded upper cylinder section 88. The tapered section 86 is shaped to mate face-to-face with the conical face 80 of the valve seat carrier piston 52. An annular groove 90 in the tapered section 86 holds an O-ring 92 sized and constructed to form a sliding seal with the cylindrical valve seat 78 at the upper portion of the central bore 76 of the valve seat carrier piston 52. A cross-bore 94 is provided at the lower part of the upper cylindrical section 88, below its internal threads.

The operating screw 54 is threaded into the lower cylindrical nose section 84 of the valve core 50 and locked, as by a chemical locking agent. It will be understood by those skilled in the art that many suitable ways of locking threaded parts together are known and may be used wherever threaded parts of the hydrant 10 are locked. The hollow valve stem 24 is threaded into the upper cylindrical nose section 88 of the valve core 50 and locked.

The operating screw 54 is threaded through the threaded opening 72 at the lower end of the valve seat carrier piston 52. Relative rotation between the valve core 50 and the valve seat carrier piston 52 will therefore move the valve seat carrier piston 52 from an open position (FIG. 4) to a closed position (FIG. 5) in which the valve core O-ring 92 enters the bore 76 of the valve seat carrier piston 52 and seals with its upper portion 78. Tightening of the valve seat carrier piston 52 with the valve core 50 is limited by mating of the conical surfaces 80 and 88.

As shown in FIG. 4, when the valve assembly 22 is open, the valve seat carrier piston 52 has moved downwardly in the bore 62 of the valve body 12 until its hex head 58 has seated on the shoulder 68 of the valve body 12. In this position, the nose 84 and conical section 86 of the valve core 50 are clear of the valve seat 78 and water is free to run from the sampling station inlet 60 through the six cylindrical openings 74 in the valve seat carrier piston 52, through its bore 76, and into the standpipe 14.

As shown in FIG. 5, when the hollow valve stem 24 is turned, the valve core 50 and operating screw 54 turn, but the valve seat carrier piston 52 is prevented from turning beyond 20° to 25° by the interaction of its hex head 58 with the protrusions 66 of the enlarged scalloped portion 64 of the bore 62 of the valve body 12. Therefore, turning the hollow valve stem 24 raises the valve seat carrier piston 52 as a piston, sealed by O-rings 56 against water flow between itself and the valve body 12, until the O-ring 92 enters the valve seat 78 and blocks flow through the valve assembly. Upward movement of the valve seat carrier piston 52 is limited by interaction of the conical surfaces 80 and 86. It will be seen that rotating the hollow valve stem 24 will move the valve seat carrier piston 52 toward and away from the valve core 48, gradually modulating flow from full open to closed.

It will also be seen that when the valve is closed, as shown in FIG. 5, the hollow valve stem 24 communicates with the standpipe 14 through the cross-bore 94.

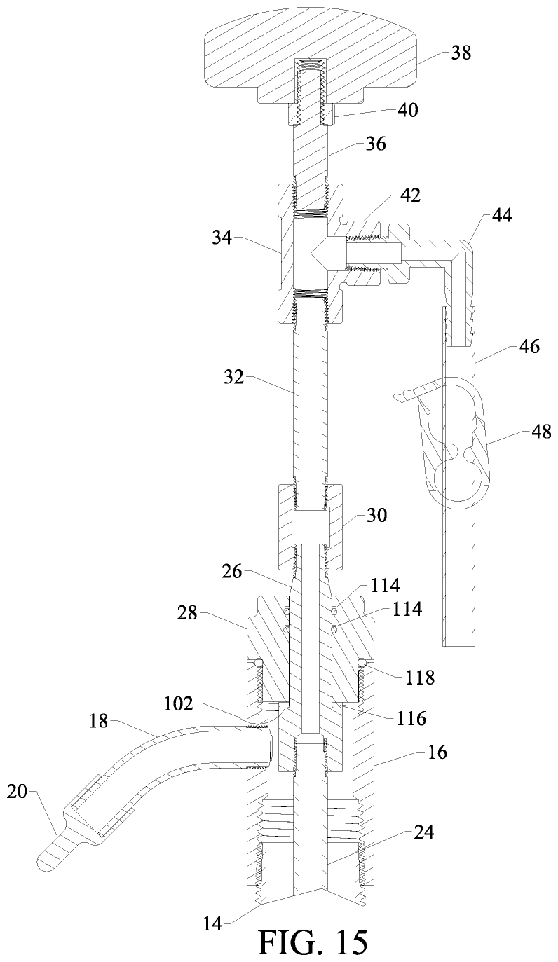

Details of the upper, above-ground, portion of the sampling station 10 are shown in FIGS. 15-19.

In FIG. 15, the drain outlet 44 is turned ninety degrees for clarity. It will be appreciated that the drain outlet 44 turns with the handle 38.

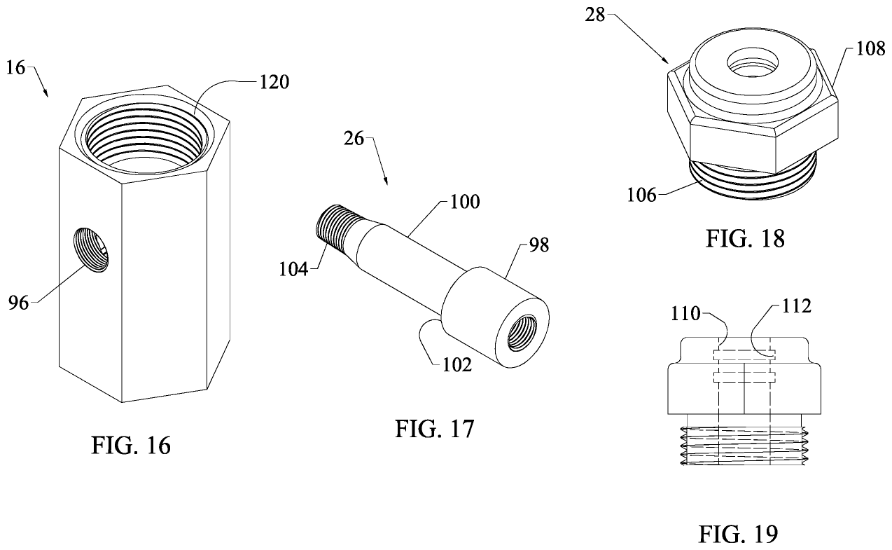

As shown in FIG. 16, the headstock 16 has internal threads at its lower and upper ends to receive the threaded upper end of the standpipe 14 and the threaded lower end of the top cap 28, respectively. A threaded opening 96 in one side of the hexagonal headstock 16 receives a threaded end of the sampling outlet 18.

As shown in FIG. 17, the connecting nut 26 includes an internally-threaded enlarged lower section 98, a cylindrical center section 100 forming a shoulder 102 with the enlarged lower section 98, and an externally threaded upper section 104.

As shown in FIGS. 18 and 19, the top cap 28 includes a lower externally threaded section 106 and a hexagonal enlarged head 108. Within a central bore 110 of the top cap 28, two annular grooves 112 seat O-rings 114.

Assembly of the sampling station 10 is straightforward.

As shown in FIG. 15, a Nylon washer 116 is slipped over the center section 100 of the connecting nut 26 and sits on the shoulder 102 of the connecting nut 26. The top cap 28 is slipped over the center section 100. The washer 116 and shoulder 102 loosely engage the bottom of the top cap 28 and limit upward movement of valve stem 20 and the valve cartridge 22 attached to the valve stem 20. The O-rings 114 set in interior annular grooves 112 in the top cap 28 engage the center section 100 of the connecting nut 26 and prevent leakage around the connecting nut 26. The threaded upper section 104 of the connecting nut 26 extends above the top cap 28, so that the coupler 30, threaded and locked onto that upper section 104, traps the top cap 28 and prevents substantial downward movement of the valve core 48 during assembly. The pipe nipple 32 is threaded and locked into the coupler 30; the tee 34 is threaded and locked onto the pipe nipple 32; and the handle-mounting rod 36 is threaded and locked into the upper end of the tee 34. The jam nut 40 and handle 38 are threaded onto the upper end of the handle-mounting rod 36 and the jam nut 40 is backed up to lock the handle 38.

As shown in FIG. 6, the valve cartridge 22 is assembled by locking the operating screw 54 into the valve core 50, then screwing it into the threaded opening 72 at the lower end of the valve seat carrier piston 52.

When preparing a sampling station 10 for a particular installation requiring a particular depth of bury, appropriate lengths of standpipe 14 and hollow valve stem 24 are chosen or are cut and threaded accordingly. The lower end of the standpipe 14 is threaded and locked into the valve body 12, and its upper end is threaded and locked into the headstock 16 with its outlet nozzle 18 and nozzle cap 20, thereby completing the fixed assembly F of the sampling station 10. The lower end of the hollow valve stem 24 is threaded into the valve core 50, and its upper end is threaded and locked into the connecting nut 26, thereby completing the removable assembly R of the sampling station 10.

An O-ring 118 (FIG. 15) is slipped into a bevel 120 at the top of the headstock 16 (FIG. 16) before assembling the removable assembly R into the fixed assembly F and tightening the top cap 28 into the headstock 16 to complete the sampling station 10.

It will be seen that in the assembled sampling station 10, the washer 116 and shoulder 102 of the connecting nut 26 engage the bottom of the top cap 28 and limit upward movement of the valve stem 24 and valve cartridge 22. When the valve assembly 22 is fully open, as shown in FIG. 4, downward movement of the valve assembly 22 and the hollow valve stem 24 is stopped by engagement of the hex head 58 of the valve seat carrier piston 52 with the shoulder 68 of the valve body 12. Therefore, further opening of the valve assembly is prevented, and the threaded operating screw 54 cannot be separated from the valve seat carrier piston 52 when the sampling station 10 is fully assembled.

Installing the sampling station 10 is simplified as compared with others. Because it has no external subterranean drain, no special subterranean drain field or drainage container is required. Because it has no external drain tube, no special precautions to avoid damaging external plumbing are required.

It will be seen that turning the handle 38 controls flow of water through the sampling station 10.

When the valve assembly 22 is open, as shown in FIG. 4, water flows from water source WS, through the curb stop CS, into the valve body 12 through the channels 74 of the valve seat carrier piston 52, through its bore 76 and valve seat 78, and through the enlarged scalloped portion 64 of the bore 62 into the standpipe 14 and through the headstock 16 and outlet nozzle 18.

When the handle 38 is turned to close the valve assembly 22, the operating screw 54 draws the valve seat carrier piston 52 upward, as the valve assembly 22 is urged upwardly by water pressure. As the valve seat carrier piston 52 rises, the valve seat 78 approaches the O-ring 92 and slows the flow of water through the sampling station 10.

When the handle is turned further, the O-ring 92 enters the valve seat 78 and stops water flow through the sampling station 10. Further tightening of the valve assembly 22 is prevented when the conical faces 80 and 86 of the valve seat carrier piston 52 and valve core 50, respectively, engage each other.

It will be seen that in both open and closed positions of the valve assembly 22, the valve assembly 22 is pushed upward by water pressure in the water system WS. Therefore, the shoulder 102 of the connecting nut 26 and washer 116 constantly bear against the lower face of the top cap 28. Furthermore, water pressure urges the valve closed.

When the valve assembly 22 is closed, and water is no longer flowing through the outlet nozzle 18, standing water in the standpipe 14 and hollow valve stem 24 may be removed by loosening the hose clamp 48 and applying a differential pressure between the outlet nozzle 18 and drain hose 46. Typically, a vacuum pump is attached to the drain hose 46; water is drawn from the standpipe 14, through the cross-bore 94 of the valve plug 50, and out the hollow valve stem 24 through the connecting nut 26, pipe nipple 32, tee 34, barb 44, and drain hose 46. Air flow will dry most or all of the remaining water, and any small amount of residual water will remain below the frost line. The hose clamp 48 and nozzle cap 20 are applied, and the sampling station 10 is freeze-proofed.

It will be seen that because the hollow valve stem 24 is concentric with the standpipe 14, water in the valve stem 24 is insulated by water in the standpipe 14. Therefore, sampling and flushing the sampling station 10 even in cold weather will not lead to flash freezing of the flushing line.

A major advantage of the construction of the illustrative sampling station 10 is that it allows for the removal of all components which may need repair or replacement merely by removing the above-ground top cap 28, without the need of any special tools. As shown in FIG. 1, the serviceable parts include the valve stem 24, and the valve cartridge 22, which includes the valve core 50, the valve seat carrier piston 52, the operating screw 54, and all of the seals (O-rings) associated with them. Should maintenance of the valve cartridge 22 be required, the curb stop CS is closed, and the top cap 28 is loosened using a standard wrench. The top cap 28 is above ground and easily accessible. Once the top cap 28 is loosened, the entire removable assembly R is simply pulled from the fixed assembly F, and O-rings 56 and 92 replaced, the parts cleaned and inspected, and any other maintenance performed.

Reinstalling the removable assembly R is straightforward. The valve is closed and then backed off a desired distance. The O-ring 118 is replaced, and removable assembly R is then inserted into the standpipe 14 and turned until the hex head 58 enters the enlarged scalloped passage 64 of the valve body 12. An operator will know that the valve seat carrier piston 52 has been fully inserted through physical feel (e.g., rotation is more difficult) or when the top cap has descended to the point of being capable of being threaded onto the headstock 16. It has been found that having three lobes or protrusions 66 is a good compromise between ease of installation and control of rotation of the valve seat carrier piston 52. The top cap 28 is tightened, and the curb stop CS is opened to complete the reinstallation of the removable assembly R.

From the foregoing, the advantages of the described system can be appreciated. Some specific advantages are described herein, and others will be apparent to those skilled in the art. All operational parts are removable from above grade after turning off the water and loosening the top cap which holds the internal components inside of the sampling station. This makes service or part replacement easier and can avoid or reduce contamination of the interior of the sampling station.

Because the entire valve assembly 22 is positioned below the frost line, damage to the components of the valve assembly by freezing is avoided, as is any major differential thermal expansion of the valve mechanism. Construction and maintenance are simplified because the valve stem 24 both actuates the valve cartridge 22 and forms a fluid path for draining water from the standpipe 14.

The structure of the valve seat carrier piston 52 allows for improved sealing between the valve seat and the valve portion. With the water (under pressure) being on the inlet side of the valve seat carrier 52, its path is through the sampling station in the direction of valve seat being pushed against the valve portion. This means that the flow of water will be “working with” the valve seat carrier piston 52 when trying to close the valve seat against the valve core 50. This closing with the water allows for the force of the pressurized system to aid in the turning off of flow and compressing the gasket (e.g., O-ring or seat rubber) for an improved seal and/or improved ease of shutting off the valve assembly.

A further advantage of the sampling station is that positioning the threaded portion of the valve stem and the valve seat carrier piston below ground reduces or eliminates freezing of an operating screw of the valve assembly by preventing or reducing residual water on the operating screw positioned above the frost line that can result in a frozen condition as in other designs. Ice on threads of an operating screw may cause friction and loss of ability to turn the screw and actuate a valve in other designs. A further advantage of the sampling station is that the valve stem is secured laterally at both the top (by the top cap) and the bottom (by the interacting between the threaded portion and the valve seat carrier and the valve seat carrier and the valve body) thus strengthening the assembly and reducing the possibility of damage to the valve seat (e.g., from lateral motion of the valve seat and/or valve portion relative to one another). Furthermore, constraining the valve stem at the top and bottom can reduce vibration from hydraulic flow as compared with devices which do not secure the bottom end of the valve stem laterally. The construction also reduces turbulent flow; this is generally advantageous and is particularly helpful when taking samples.

Additionally, the valve assembly as described is superior to the use of other valve types such as ball valves. Ball valves are designed to be utilized in the full open and the full closed position and using them to throttle the flow can lead to faster wear and need for replacement of the ball valve. Leaving them in the closed position for extended durations at high pressures can result in the seat where the ball lands to deform and leak making it more likely that the seat could be cut or damaged. Another concern when using ball valves is that debris on the ball before operation can cause scratching of the ball or seats increasing the likelihood that replacement will be needed. Therefore, the valve portion and valve seat and valve seat carrier of the type described herein avoids these deficiencies of ball valves and therefore provides benefits of enhanced durability and capability. In the case of a failed ball valve, repair or replacement would require a full dig up and removal of the station itself as they aren't repairable from above ground. The sampling station as described herein does not use a ball valve and is also repairable above ground through the removal of the relevant components as described herein.

Another advantage of the sampling stations described herein is that by having a combined valve operation and drain tube that is positioned internally to the standpipe, as opposed to a separate and external liquid evacuation line, the water flowing through the standpipe during sampling warms the combined valve operation and drain tube as it is from below the frost line. This warming reduces or prevents ice formation within the drain tube thereby preserving its function by reducing or eliminating the formation of blocking or damaging ice, especially as compared to drain tubes positioned externally to a standpipe or other sampling pipe.

A person of skill in the art can appreciate further advantages provided by the disclosed systems and methods.

It will be noted that although the valve stem is restrained from vertical motion, some amount of play is provided to allow easy rotation of the valve stem. Further, even if the valve stem were to move vertically downward and the valve seat carrier were to remain stationary as the valve is closed, the slidable piston 51 would nonetheless continue to make the valve cartridge removable without tools once the top cap was removed.

Changes can be made in the above constructions without departing from the scope of the disclosure. It is intended that all matter contained in the above description or shown in the accompanying drawings shall be interpreted as illustrative and not in a limiting sense.

For example, and without limitation, the valve mechanisms and other components described herein are described with reference to use in a sampling station but can be used in other applications such as other hydrants (flushing, fire, snow, sanitary, etc.) or in entirely different applications. In alternative constructions, the valve core (plug) may be positioned below the valve seat piston. The plug portion may comprise a conventional elastomeric plug rather than O-ring 92. Although the illustrative embodiments eliminate the need for a below-grade drain, many of the features of the invention can be incorporated into hydrants that have a subterranean drain. Many of the components that are described as pipes, lines, conduits, and the like can be of circular cross-section, polygonal cross-section, or have any other suitable shape or structure. Different handles may be provided, positioned above or below the drain outlet. In embodiments, the drain outlet itself acts as a handle. The embodiments described herein have center line axially aligned components, but these components need not be aligned with the centerline. Other further embodiments are possible consistent with this disclosure and the following claims.

Many of the novel features of the invention may be used by themselves without incorporating others. For example, the hollow valve stem used for evacuating the standpipe may be used without the inside-out valve or without a complete valve assembly below the frost line.

These examples are merely illustrative.

1. A hydrant comprising:
a conduit having a lower end in fluid communication with a liquid source;
a valve assembly slidably mounted in the conduit, the valve assembly comprising a valve seat portion and a plug portion sized and constructed block flow through the valve seat portion, hence through the conduit, and a threaded connector connecting the valve seat portion to the plug portion;
a valve stem attached to the valve assembly and extending upward through the conduit, the valve stem being attached to one of the valve seat portion and the plug portion and enabling relative rotation between the valve seat portion and the plug portion; and
a removable above-ground stop, the removable stop being sized and shaped to limit upward movement of the valve stem; disabling the stop enabling sliding the valve assembly upward out of the conduit.

2. A hydrant in accordance with claim 1, wherein the valve stem is constrained, relative to the conduit, in a vertical direction such that the valve stem is limited in movement along an axis in the vertical direction and wherein the valve seat is limited in rotational movement.

3. A hydrant in accordance with claim 2, wherein the valve stem is retained in the conduit and constrained in an axial direction by a top cap, the top cap being releasably attachable to the conduit.

4. A hydrant in accordance with claim 3, wherein the conduit comprises a headstock, a discharge nozzle is coupled to the headstock and in fluid communication with the conduit through a channel of the headstock, and the top cap is attached to the headstock.

5. A hydrant in accordance with claim 4, wherein the valve stem extends through the headstock, and wherein a handle is attached to the valve stem above the headstock to permit rotation of the valve stem and actuation of the valve assembly through the rotation of the valve stem.

6. A hydrant in accordance with claim 5, wherein the valve stem is hollow and a valved opening in fluid communication with the valve stem is positioned above the handle.

7. A hydrant in accordance with claim 2, wherein the conduit is a standpipe, the valve assembly comprising:
a valve body coupled to the first end of the standpipe, the valve body including a channel therein;
a valve portion of the valve stem and a threaded portion of the valve stem extending downward from the valve portion; and
a valve seat carrier having a through bore, the valve seat carrier having a valve seat adapted and configured to engage with the valve portion of the valve stem to form a seal when the valve assembly is in the closed position, the through bore extending through the valve seat, the valve seat carrier having a threaded portion of the through bore adapted and configured to engage with the threaded portion of the valve stem, and the valve seat carrier having at least one channel in fluid communication with the through bore extending through the valve seat such that liquid can travel through the channel and the through bore and through the valve seat carrier when the valve assembly is in the open position and liquid cannot pass through the through bore when the valve seat and the valve portion form a seal,
wherein the valve seat carrier and the valve body are adapted and configured to prevent rotation of the valve seat carrier when positioned inside the valve body such that rotation of the valve stem and the associate threaded portion causes the valve seat carrier to raise or lower depending on the rotation direction of the valve stem because of the interaction between the threaded portion of the valve stem and the threaded portion of the through bore of the valve seat carrier.

8. A hydrant in accordance with claim 7, wherein the valve body includes a scalloped portion and wherein the valve seat carrier includes a polygonal portion adapted and configured to engage with the scalloped portion of the valve body such that rotation of the valve seat carrier is prevented by the interaction between the scalloped portion of the valve body and the polygonal portion of the valve seat carrier.

9. A valve assembly comprising, a valve body in fluid connection with a conduit and a valve cartridge assembly adapted to be inserted into and removed from the valve body through the conduit,
the cartridge assembly comprising a valve seat body, a valve plug body, and a threaded connection between the valve plug body and the valve seat body,
the valve seat body forming a water-tight sliding fit with the valve body, at least a portion of the valve body and at least a portion of the valve seat body being non-circular to limit rotation of the valve seat body relative to the valve body,
the valve plug body being freely rotatable through at least 360° and being limited in axial movement, rotation of the valve plug body moving the valve seat body into and out of a closed position blocking flow of liquid through the cartridge assembly.

10. The valve assembly of claim 9 wherein the valve plug body comprises a valve stem extending through the conduit, the plug body being restrained from axial movement by the valve stem

11. The valve assembly of claim 10 wherein the conduit comprises a pipe, the valve body being threaded to a distal end of the pipe, and wherein the valve stem is restrained from movement away from the distal end of the pipe.

12. The valve assembly of claim 11 wherein the valve stem is restrained by a head stock assembly and wherein removal of at least a part of the head stock assembly permits pulling the cartridge by the valve stem from the pipe.

13. The valve assembly of claim 9 wherein a cylindrical portion of the valve seat body comprises at least one O-ring forming a sliding seal with a cylindrical portion of the valve body.

14. A hydrant having a standpipe with an above-ground operational outlet, a subterranean valve, and a valve stem operating the valve, the valve stem extending upward from the valve through the standpipe, wherein the valve stem is hollow, the valve stem having a subterranean inlet and an above-ground flushing outlet in fluid connection with the inlet, whereby liquid can be evacuated from the standpipe and the hollow valve stem by attaching at least one of a vacuum source or a pressure source to the above-ground flushing outlet while the above-ground operational outlet is open.

15. The hydrant of claim 14 wherein the subterranean inlet of the valve stem is above the subterranean valve.

16. The hydrant of claim 14 wherein the above-ground outlet of the valve stem comprises a manually-operable closure.
